(19) United States
(12) Patent Application Publication
(10) Pub. No.: US 2022/0280094 A1
(43) Pub. Date: Sep. 8, 2022

(54) KIT COMPRISING IMPLANTABLE, FLEXIBLE MULTI-LEAD CARDIAC MONITOR WITH OPEN-CIRCULAR SHAPE AND IMPLANTATION TOOL TO ACCOMMODATE REVERSIBLY SAID MONITOR

(71) Applicant: BERNE UNIVERSITY OF APPLIED SCIENCES, Biel (CH)

(72) Inventors: Martin CLEMENT, Cossonay (CH); Armando WALTER, Bevaix (CH); Thomas NIEDERHAUSER, Niederwangen b. Ben (CH)

(73) Assignee: BERNE UNIVERSITY OF APPLIED SCIENCES, Biel (CH)

(21) Appl. No.: 17/753,237

(22) PCT Filed: Aug. 27, 2020
(86) PCT No.: PCT/EP2020/073994

§ 371 Date: Feb. 24, 2022

(30) Foreign Application Priority Data
Aug. 29, 2019 (EU) 19194460.2

(51) Int. Cl. A61B 5/333; A61B 5/00; A61B 5/29; A61B 17/34; A61B 17/3211; A61B 5/287; A61B 5/263; A61B 5/308

(52) U.S. Cl. CPC A61B 5/333; A61B 5/0006; A61B 5/0031; A61B 5/29; A61B 17/3468; A61B 17/3211; A61B 5/0017; A61B 5/287; A61B 5/263; A61B 5/308; A61B 2562/164; A61B 2562/166; A61B 2560/0219; A61B 2017/00853

(57) ABSTRACT

A kit for implanting a flexible multi-lead cardiac monitor for recording biosignals when the monitor is placed under the skin. The kit includes the monitor, an implantation tool for implanting the monitor under the skin, and optionally a surgical knife. The monitor exhibits an open-circular shape, is based on a flexible printed circuit board with at least two sensing electrodes, optionally a ground electrode, includes a main circuit based on the FPCB, and is free of a casing. The implantation tool exhibits an open-circular shape to reversibly receive the monitor, includes a base to reversibly accommodate the monitor, a handle connected to the base, and a slider reversibly insertable into the base. A process to make the monitor of the kit, the monitor obtainable according to the process, a process to monitor biosignals with the monitor, and the use of the kit, of the monitor and of the implantation tool.

### CROSS REFERENCE TO RELATED APPLICATION

This application is a national stage entry of PCT/EP2020/073994 filed Aug. 27, 2020, under the International Convention claiming priority over European Patent Application No. 19194460.2 filed Aug. 29, 2019.

### FIELD OF THE INVENTION

The present invention relates to a kit suitable for implanting an implantable, flexible multi-lead cardiac monitor for recording biosignals over years when the monitor is placed under the skin of a living body, the kit is includes the monitor and an implantation tool for implanting the monitor under the skin, a process to make the monitor, an implantable, flexible multi-lead cardiac monitor obtainable according to the process to make the monitor, a process to monitor biosignals with the monitor of the kit and the monitor obtainable according to the process, as well as the use of the kit, the monitor and the implantation tool.

### BACKGROUND OF THE INVENTION

With the aging of the population worldwide and the increasing of the obesity rate, cardiovascular diseases (CVD), such as cerebrovascular stroke, myocardial infarction or heart rhythm disorders, i.e. cardiac arrhythmias, are increasingly spread in western countries. Therefore, the need for performing diagnosis required for proper cardiovascular treatment at reduced costs is at the top of the priorities of health care provider.

ECG analysis is among the most relevant methods to detect and diagnose a number of heart rhythm disorders. The ECG, i.e. the monitoring system, collects the electrical signals emitted by the heart's electrical system undergoing typical sequences of cell depolarization and repolarization. ECG measurements are typically performed by a physician in a medical facility within minutes. Long-term ECG recording for 24 to 48 hours, i.e. Holter monitoring to detect rare and short arrhythmias such as paroxysmal atrial fibrillation or even up to 30 days for patients suffering a cryptogenic stroke where the etiology is unknown, repetitive or event-triggered ECG monitoring may be performed, are also established methods.

For patients suffering from transient cardiac arrhythmia, however, the time span of such long-term ECG measurements is still too short. Furthermore, in some cases the patient does not even feel the episodes, which makes ECG measurements right after the occurrence impossible. In order to overcome this, implantable ECG-recorders—so-called event recorders—are suggested to record ECG for up to three years after being implanted into the patient. Known event recorders have a rigid metal case, a length of about 4 cm and most typically a linear form, i.e. they are one-dimensional-vector recorders with one signal and one ground electrode. The data measured of such recorders suffer from a relatively poor signal quality and—due to the limited capacity of the battery—they do not record ECG signals continuously, but only events, i.e. only the abnormal ECG sequences. As a result of recording events only, ECG sequences well before and after the events are not stored and thus, they cannot be analyzed.

During the depolarization and repolarization of myocytes of the heart, an electric field is generated, which can be measured. Willem Einthoven found that when electrodes are placed at specific locations, i.e. the two arms and one of the legs, improved measurement data are obtained. Said locations form the corners of the so-called Einthoven's triangle. Thus, when implanting ECG-recorders, the electrodes of the recorders should ideally be placed to fit within the Einthoven's triangle. This, however, is only possible with Holter Monitors during long-term ECG recording, which are external and thus not implantable monitors. Today's implantable recorders, known as Implantable Loop Recorders (ILR), are not capable of measuring Einthoven's triangle, even when implanted during a conventional surgery.

WO-A-2011084450 describes biomedical devices and methods of making and using biomedical devices for sensing and actuation applications, such as flexible and/or stretchable biomedical devices, electronic devices useful for establishing in situ conformal contact with a tissue in a biological environment. Furthermore, implantable electronic devices and devices administered to the surfaces(s) of a target tissue, such as for obtaining electrophysiology data from a tissue, e.g. cardiac, brain tissue or skin. The implantation of such devices requires a conventional surgery, but it is not possible with minimally invasive implantation techniques. Furthermore, said devices are very thin in order to be conformal with the tissue. Thus, they lack stability. FPCB's and an implantation tool are, among others, not mentioned. To position electrodes within the Einthoven's triangle is not possible.

US-A-2007/0016089 describes an implantable medical device for subcutaneous implantation within a human being. The implantable medical device includes a pair of electrodes for sensing electrical signals from the human being's heart. Electronic circuitry having digital memory is provided with the electronic circuitry designed to record the electrical signals from the heart. The electronics of the electronic circuitry is housed in a case having a tapered shape to facilitate implantation and removal of the implantable medical device. The monitor includes leads of different lengths that are attachable to a shell housing having an exterior and an interior. Thus, the monitor comprises connections of said leads at various places, such as screws. Such connections, however, are vulnerable and subject to disruption. Although the monitor may be bended, the limited bending does not allow an arrangement of three or more electrodes to from a triangle to make use of the Eindhoven's triangle for long-term ECG measurements. Furthermore, an implantation tool to implant the monitor is not disclosed.

US-A-2010/0331868 discloses a method for constructing an instrument with a two-part plunger for subcutaneous implantation. An incising body is formed by defining a non-circular coaxial bore and sharpening a distal bottom edge. A two-part plunger including a tongue blade assembly and a plunger assembly is constructed. The two-part plunger is inserted through an end of the incising body. The implantation instrument has a straight or a curved incising shaft. The curved instrument has only a minor bending, while the construction of the instrument would not allow to insert an implant with a bending of e.g. 90° or more. Furthermore, the implant is small in size and exhibits—if any—only a slight bending. Thus, the implant is not suitable for placing three or more electrodes which may form a triangle to make use of the Eindhoven's triangle for ECG measurements.

### SUMMARY OF THE INVENTION

Therefore, there is a need to overcome said disadvantages of the present ECG-recorders. Thus, there is a requirement for a recorder for long-term ECG measurements which can be implanted easily with the minimally invasive technique. Furthermore, the ECG-recorder must, when implanted, provide good ECG signal quality over years by making use of the Einthoven's triangle. The recorder shall record ECG signals also before and after an event, and in the best case continuously. It shall be easy to implant and—when implanted—it shall not bother the patient. Additionally, it should be possible to read out acquired data whenever required and ideally to recharge the battery of the implanted recorder in a non-invasive way to increase the number of acquired data and/or to extend the time the recorder can acquire data.

Surprisingly, it was found that these requirements can be fulfilled with a kit suitable for implanting an implantable, flexible multi-lead cardiac monitor (1) which is suitable for recording biosignals over years when the monitor (1) is placed under the skin, the kit includes the monitor (1), an implantation tool (6) for implanting the monitor (1) under the skin, and optionally a surgical knife, wherein:

the monitor (1) exhibits an open-circular shape, is based on a flexible printed circuit board (FPCB) (2) with at least two sensing, preferably at least three, electrodes (3) and optionally a ground electrode (4), wherein the monitor (1) comprises a main circuit (5) based on the FPCB (2), and wherein the monitor (1) is free of a casing; and

the implantation tool (6) exhibits an open-circular shape to receive the open-circular monitor (1) reversibly, the implantation tool (6) includes a base (61), which is suitable to accommodate the monitor (1) reversibly, handle (62), which is connected to the base (61), to hold and position the implantation tool, and

slider (63), which is insertable into the base (61) reversibly and thus capable to push the accommodated monitor (1) out of the implantation tool (6) to the final position,

wherein the base (61) includes a base bottom (61a), base sidewalls (61b) and a base surface which is at least partially open to allow the slider (63) to slide along the base bottom (61a) to push the accommodated monitor (1) out of the base (61) wherein the base bottom (61a), the base sidewalls (61b) and the base surface are preferably arranged along the open-circular shape of the implantation tool (6).

Also, there is a process to make the monitor (1) of the kit according to the invention, wherein:

the at least two sensing electrodes (3), the base (5a) of the main circuit (5) and the optional ground electrode (4) are made from the same flexible printed circuit board (FPCB) (2),

the amplifier (5b), the controller (5c), the optional battery (5d), the memory (5e), and the optional transmitter or transceiver (5f), the optional DC-restorer (5g), and/or the optional feedback circuit (5h) are connected to the base (5a) and thus to the main circuit (5), preferably by soldering, welding, bonding, and/or gluing; and

optionally at least the side of the FPCB (2), which is opposite to the electrodes (3, 4), is coated with the dielectric coating (28).

Furthermore, there is also is the implantable, flexible multi-lead cardiac monitor (1) obtainable according to the process to make the monitor (1) according to the invention.

Also, there is is a process to monitor biosignals with the monitor (1) of the kit according to the invention, and/or the monitor (1) obtainable according to the process to make the monitor according to the invention, wherein:

the biosignals are measured, preferably continuously, with the sensing electrodes (3), the thus obtained data are stored on the main circuit (5), in particular in the memory (5e) of the main circuit (5), wherein preferably all measured data are stored until the data are read out, and

the measure and stored data are read out via the transmitter or transceiver (5f), wherein the transmitter or transceiver (5f) comprises an antenna, using a reader by wireless communication, preferably by RFID, and/or optical wireless communication such as NIR.

Also, there is the use of the kit according to the invention to implant the monitor (1) of the kit and obtainable according to the process of the invention to make said monitor (1) with the implantation tool (6) of the kit according to the invention.

In addition, there is the use of the monitor (1) of the kit according to the invention and of the monitor (1) obtainable according to the process of the invention to make said monitor (1) for long-term cardiac monitoring over years, in particular when the monitor (1) is implanted under the skin.

Furthermore, there is the use of the implantation tool (6) of the kit according to the invention to accommodate reversibly the monitor (1) of the kit according to the invention and/or the monitor (1) obtainable according to the process of the invention to make said monitor (1).

### BRIEF DESCRIPTION OF THE DRAWINGS

FIG. 1 illustrates a non-limiting and schematic scheme of an implantation tool according to the present invention;

FIG. 2 illustrates a non-limiting and schematic view of the monitor according to the present invention;

FIG. 3 illustrates a non-limiting embodiment of the slider according to the present invention;

FIG. 4a illustrates a non-limiting example of the open end of the base of the implantation tool according to the present invention;

FIG. 4b illustrates the front-view at the open end of the base from the implantation tool according to the present invention;

FIG. 5 shows an exemplary cross section of the FPCB according to the present invention; and

FIG. 6 shows a non-limiting block diagram of the main circuit on the FPCB according to the present invention.

### DETAILED DESCRIPTION OF THE INVENTION

Surprisingly, the kit includes the monitor (1) and the implantation tool (6), the process to make the monitor (1), the monitor (1) obtainable according to the invention, the process to monitor biosignals with the monitor (1) as well as the use of the kit, of the monitor (1) and of the implantation tool (6) exhibit many advantages.

Due to the open circular shape of the kit according to the invention, and thus of the monitor (1) and the implantation tool (6), a multitude, i.e. at least 2, preferably at least 3, of sensing electrodes (3) can be placed on one and the same surface of the monitor (1) with sufficient distance from each other. Therefore, after the monitor (1) is implanted, those electrodes (3) can be selected for biosignal measurements, in particular for ECG measurements, which are optimally located, i.e. placed, to fit optimally with the Einthoven's triangle. Thus, optimal data output with best ECG signal quality is provided over years. Hence, it is surprisingly possible to implant through just a small cut in the skin of e.g. a few centimeters, i.e. by minimally invasive implantation, a monitor (1) with a diameter of e.g. 20 cm or more. In case the monitor comprises more than 3 electrodes (3), the monitor may be equipped with a suitable algorithm to select the optimal electrodes (3) for ECG measurement. Alternatively, the electrodes (3) are configured externally after implantation, or the ECG signal from all electrodes (3) are measured and stored. Thus, the skilled person in the art is well capable to place the monitor (1) during the implantation step into the optimal body's location. Furthermore, he can select to best placed electrodes (3) for ECG measurement, in case the monitor (1) comprises more than 3 electrodes (3).

Since no casing is needed to host the monitor (1), the battery of the implanted recorder can be recharged non-invasively, which is highly advantageous. Thus, it is possible to acquire data continuously and to read out said data whenever required. Hence, it is possible to record ECG signals also well before and after arrhythmic events, such as atrial fibrillation.

The monitor (1) according the invention is unexpectedly—due to its open-circular shape and the flexible printed circuit board (FPCB) the monitor (1) is based on—a flexible monitor, i.e. it does not have a rigid form and thus it adapts easily to the environment, e.g. to the tissue and/or muscles, it is implanted into. Due to the increased flexibility, the size of the monitor (1) can be increased, which itself helps to increase the biosignal quality, in particular the ECG signal quality. Hence, a monitor (1) with a diameter of 7 cm can be bent with little force, e.g. with 0.1 N or less, to an angle of 45° or more. Additionally, the open-circular shape allows easily to place two sensing electrodes (3) and one ground electrode (4), wherein the electrodes (3, 4) can be placed e.g. to form the corners of an equilateral, isosceles or right-angled triangle, to provide optimal distance between the electrodes. Thus, the monitor (1) forms at least a two-dimensional-vector monitor with two or more sensing electrodes (3) and one ground electrode (4), wherein the electrodes (3, 4) have direct contact to the tissue and/or muscles of the living body. Hence, such a two-dimensional-vector monitor (1) provides increased sensing quality and redundant data. Furthermore, the open-circular shape of the monitor (1) allows the making of monitors (1) with larger surfaces while the implantation cut remains small. This allows to place batteries with bigger capacities onto said monitors (1). As a conclusion thereof, the monitor (1) can record biosignals such as ECG signal continuously, i.e. the monitor (1) can be used to measure—and save—ECG signals of the arrhythmic event as well as well before and after the arrhythmic event. Hence, diagnosis of the patient's disease is improved compared to purely event-triggered recording. Since the monitor (1) is a flexible monitor, the monitor (1) adapts easily—when implanted—to the body and its motions, e.g. when moved.

Since the monitor (1) is based on FPCB (2), the electrodes (3, 4) as well as the main circuit (5) can easily be integrated into the monitor (1). Thus, no connections and/or cables between the electrodes (3, 4) and the main circuit (5) are required. As a conclusion thereof, a particularly stable monitor (1) is thus obtained, which is not prone to defects. Furthermore, since the monitor (1) is based on FPCB, it is after implantation not only well compatible with its surrounding tissue, but the FPCB facilitates also the implantation of the monitor (1) with the implantation tool (6) by turning.

The monitor (1) is free of a casing and thus the monitor (1)—and in particular the electrodes (3, 4)—provide improved signal quality. Furthermore, the battery can be recharged easily and non-invasively.

The implantation tool (6) according to the invention includes the base (61), the handle (62) and the slider (63) is a tool which i) is dedicated to accommodate the monitor (1) in the base (61), ii) facilitates to implant the monitor (1) under the skin of a living body surprisingly by simply turning the tool (6), by turning the handle (62) by hand in one direction, and thus to insert the monitor (1) through a cut under the skin, iii) to push the accommodated monitor (1)—when under the skin—with the slider (63) out of the base (61) to the final position, and iv) finally remove the base (61) and the slider (63) out of the cut by a reverse-turn of the handle (62). Thus, the implantation tool (6) allows an easy and straight forward implantation of the monitor (1) under the skin of a living body through a small cut, i.e. through a cut with a diameter much smaller than—e.g. just 10 to 20% of—the diameter of the implanted monitor (1).

The process to make the monitor (1) according to the invention is surprisingly a straightforward process well established in the semiconductor industry. Thus, it requires less production steps than the today commercially available recorders. Furthermore, the monitor (1) with the electrodes (3, 4) and the transmitter or transceiver (5f), preferably including an antenna, is not shielded by a metal case. Thus, the transmitted and/or received electromagnetic signals are less attenuated which leads to an extended range and flexibility of the monitor (1). Furthermore, the monitor (1) provides an increased MRI-compatibility, which is highly advantageous. Thus, the monitor (1) according to the invention does not comprise a rigid metal case, but to the contrary, it is a flexible monitor (1) allowing to adapt to the body and its motions.

The process to monitor biosignals with the monitor (1) according to the invention allows to monitor biosignals such as ECG signals continuously, i.e. the monitor (1) can unexpectedly be used as a continuous recorder, which is a big advantage over event-triggered or loop recorders. Hence, ECG signals of the arrhythmic event as well as well before and after the arrhythmic event can be recorded, saved and later on analyzed. Thus, diagnosis of the patient's disease is improved compared to pure event-triggered recording.

The use of the monitor (1) according to the invention allows long-term cardiac monitoring over years with superior signal quality and redundant data.

The use of the implantation tool (6) according to the invention provides an easy-to-use receptacle for inserting the monitor (1) into the tool (6) and for implanting the monitor (1) under the skin of a living body.

### The Kit

The kit according to the invention is particularly suitable for implanting the flexible multi-lead cardiac monitor (1) with the help of the implantation tool (6)—and the optional surgical knife—by minimally invasive implantation techniques. The monitor (1) itself is thus easy to implant and does not—due to its flexibility and flat structure—bother the patient, after it is implanted.

The kit includes the monitor (1), the implantation tool (6) and the optional surgical knife are preferably sterile packed to be ready for use.

Both, the monitor (1) and the implantation tool (6) of the kit exhibit an open-circular shape. While said shape enables the monitor (1) to arrange the sensing electrodes (3) in an optimal manner, e.g. to form the corners of a equilateral, isosceles or right-angled triangle, the open-circular shape of the implantation tool (6) enables to receive the open-circular monitor (1) reversibly.

The term open-circular shape, i.e. the shape of the monitor (1) and the implantation tool (6) reveals the shape of a circle, i.e. with a circumference of a circle with an angle of 360°, or of a section of a circle, i.e. of an angle of less than 360°, preferably of an angle of between 180° to 320°. Furthermore, the open-circular shape may exhibit a circumference of a circle with an angle of more than 360°, preferably up to 400°. Hence, the two end-regions of the monitor (1) and the implantation tool (6) may be overlapping. However, in any case the circular shape of the monitor (1) is not a closed shape but has two ends. As such—and due to the flexible printed circuit board (FPCB) (2) it is based on—the ends of the monitor (1) are bendable and thus may form a section of a spiral.

In a preferred embodiment, the open-circular shape of the monitor (1) and of the implantation tool (6) exhibits a circumference of an angle of between 90° and 400°, preferably between 180° to 320°, to allow placement of at least three sensing electrodes (3) at corners of an equilateral, isosceles or right-angled triangle have an angle of between 60° and 90°, in particular between 75° and 90°.

In another preferred embodiment, the outer diameter of the monitor (1) with the open-circular shape ranges from 3 to 20 cm, preferably from 4 to 15 cm, and in particular from 4 to 10 cm; and/or the inner diameter of the monitor (1) ranges from 2 to 18 cm, preferably from 3 to 12 cm, and in particular from 3 to 8 cm. Thus, the outer diameter of the base (61) of the implantation tool (6) is somewhat larger and the inner diameter of the base (61) of the implantation tool (6) is somewhat smaller than the dimensions of the monitor (1) to allow the monitor (1) to slide well inside the base (61).

The width of the strand of the monitor (1) is half of the difference of the outer to the inner diameter of the monitor (1). Thus, it may range from 0.1 mm to about 3 cm, preferably from 0.5 cm to 2 cm. This allows a size of electrodes (3, 4) of up to about 5 mm² or more, wherein the electrodes may be of e.g. circular or rectangular shape.

The thickness of the monitor (1)—measured in the vertical to the diameter of the open-circular shape measured according to DIN 50986—may vary from 0.5 mm to 5 mm, preferably from 1 to 3 mm.

The dimensions of the implantation tool (6), in particular the base (61) of the implantation tool with its base bottom (61a) and base sidewalls (61b), are designed to receive the monitor (1) and thus the outer diameter is slightly larger and the inner diameter of the implantation tool (6) is slightly smaller than the monitor (1) itself. The skilled person is knowledgeable to adjust to optimal dimensions of the implantation tool (6).

In order to implant the monitor (1) under the skin of a living body, the skin is cut at the desired place, most typically in the heart region. Cutting the skin may be performed with a surgical knife. Alternatively, the open end of the implantation tool (6) may form a blade, e.g. made from stainless steel, to make the cut. In a second step, the open end of the implantation tool with the incorporated monitor (1) is inserted into the cut and the implantation tool (6) with the monitor (1) is pushed under the skin by turning the handle (62) in the respective direction. When all of the monitor (1) is implanted, i.e. inserted under the skin, the slider (63) is fixed e.g. with the another hand and the base (61) with the handle (63) of the implantation tool (6) are turned reversely, thus turning them out of the body. The slider (63), however, remains at its place to avoid that the monitor (1) slips out, e.g. together with the base (61). In case the monitor (1) further contains a barbed hook, the latter will assure that the monitor (1) will stay at its last position and not slip out of the body. The slider (63) is then finally removed together with the remaining part of the base (61) of the implantation tool. Thus, it is surprisingly possible to implant through a small cut in the skin, i.e. by minimally invasive implantation, e.g. of just a few centimeters, a monitor (1) with an outer diameter of up to 20 cm or more.

The monitor (1) the Flexible Printed Circuit Board (FPCB) (2) and the Main Circuit (5)

The term monitor (1) refers according to the invention to the monitor (1) of the kit as well as to the monitor (1) obtainable according to the process to make the monitor (1) of the kit, i.e. the monitor (1) obtainable according to the process to make the monitor (1).

The implantable, flexible multi-lead cardiac monitor (1) is designed for measuring biosignals under the skin, i.e. subcutaneous, of a living body, in particular when implanted in the area of the heart, for long-term measurements of said biosignals over years.

The biosignals to be monitored arise most typically from the heart region and relate to electrical biosignals. They are understood—according to the invention—to stand for the time-varying bioelectrical signals which are measured and have diagnostic potential. They usually refer to the change in electric current produced by the sum of an electrical potential differences across a specialized tissue, organ or cell system like the myocytes of the heart. Thus, the biosignals may be measured by electrocardiography (ECG), electroencephalography (EEG), electromyography (EMG), or other biosignals from plethysmography, impedance, and/or temperature measurements. A preferred method to measure biosignals is electrocardiography (ECG).

The term living body is understood—according to the invention—to stand for a living human body as well as living animal bodies, in particular mammals, having a certain minimal size. The skilled person can easily evaluate the minimal size required of the human or animal.

The monitor (1) exhibits an open-circular shape, is based on a flexible printed circuit board (FPCB) (2) with at least two sensing, preferably at least three, electrodes (3) and optionally a ground electrode (4). The monitor (1) further comprises a main circuit (5) based on the FPCB (2). In addition, the monitor (1) is free of a casing to allow the FPCB—and thus the monitor (1)—direct contact with its environment, in particular after it is implanted.

The monitor (1), i.e. recorder, is a multi-lead monitor (1), i.e. it comprises at least two, preferably at least 3, or even 6 or more, in particular 12 or more, sensing, i.e. signal or lead, electrodes (3) and optionally a ground electrode (4). Thus, 3 sensing electrodes (3) allow to place the electrodes within the Einthoven's triangle. When the monitor (1) comprises more than 3 sensing electrodes (3), it is possible to select those electrodes (3) which fit best into Einthoven's triangle.

Furthermore, the monitor (1) is a flexible monitor. The term flexible is understood to stand for materials having a deflection of at least 1 cm when a material having a linear length of 10 cm is subjected to a force of 0.1 N. It is noted that the thickness and the width are irrelevant. Thus, a more rigid material may be flexible according to the invention, if the thickness and the width are sufficiently small to allow the deflection. However, a twistable material may be regarded as inflexible if it is too thick and/or too broad to allow said deflection.

The monitor (1) is based on a flexible printed circuit board (FPCB) (2) with at least two sensing electrodes (3) and optionally a ground electrode (4). Furthermore, the monitor (1) comprises a main circuit (5).

In a preferred embodiment, one end of the monitor (1) comprises a barbed hook, like a harpoon. This allows the monitor (1) to stay during and right after implantation at its place and thus it does not slip out again. When the harpoon is made of a bioresorbable material such as magnesium, the harpoon deteriorates within days, weeks or months and thus does not hinder the explantation of the monitor (1) when the time is due.

The monitor (1) according the invention is based on a flexible printed circuit board (FPCB) (2). The FPCB (2) comprises the at least two sensing, i.e. signal or lead, electrodes (3) and the optional ground electrode (4), i.e. the electrodes (3, 4) are integrated—and thus part of—the FPCB (2).

The ground electrode (21) is optional, although it is in general recommended to obtain improved signal quality.

In one preferred embodiment, the monitor (1) comprises at least three, preferably 6 or more, in particular 12 or more, sensing electrodes (3), wherein the sensing electrodes (3) and the optional ground electrode (4) are integrated into the flexible printed circuit board (FPCB) (2), wherein the sensing electrodes (3) are arranged to form the corners of a polygon, in particular an equilateral, right-angled or equiangular polygon, and wherein the sensing electrodes (3) may comprise a pre-amplifier or buffer (3a). The optional ground electrode (4) serves as reference and improves signal quality, if present. The electrodes (3, 4) are dry electrodes. This arrangement provides optimal distance between the electrodes and thus increased signal quality. Additionally, the signals received provide further information for improved and extended data interpretation. Furthermore, the monitor (1) does not exhibit interfaces between the FCBP (2) and the electrodes (3, 4) and thus it cannot break apart in between when used according to instructions.

Non-limiting examples of a suitable preamplifier or a buffer (3a) include dedicated inverting or non-inverting operational amplifier (opamp) circuits or in the case of the buffer, voltage follower circuits built using opamps or metal oxide semiconductor field effect transistor (MOSFET) circuits.

In another preferred embodiment of the monitor (1), the flexible printed circuit board FPCB (2) is a layered composite material including a first conductive material layer (21) capable to act as electrodes (3, 4), a first dielectric layer (22), a signal layer (23), optionally an adhesive layer (24), a further dielectric layer (25), an optional further signal layer (26), and/or an optional solder mask layer (27), wherein optionally at least the side of the FPCB (2), which is opposite to the electrodes (3, 4),—or even the total surface of the monitor (1) except the surfaces of the electrodes (3, 4), which may be preferred—may be coated with a dielectric coating (28). These layers may be arranged in said order. However, they may be in any order, as long as the conductive layers (21, 23, 26) are separated from each other by a dielectric layer (22, 25). Furthermore, the FPCB (2) may comprise additional layers, e.g. further signal layers and dielectric layers. The skilled person is aware of the optimal FPCB for specific uses and he can make the selection. Additionally, he is also capable of manufacturing suitable FPCB's. At least the side of the FPCB (2), which is opposite to the electrodes (3, 4), may be coated with a dielectric coating (28). The dielectric coating (28) comprises preferably at least a silicon oxide (SiOₓ), an organic silicon oxide such as polydimethylsiloxane (PDMS), a Parylene-type material, copolymers thereof, and/or block-(co)polymers thereof. Parylene-type materials, such as Parylene C, Parylene D, Parylene HT or Parylene N, are inert, hydrophobic polymeric coating materials based on Poly-p-xylylene, and/or halogenated polymers thereof. A preferred coating comprises Parylene C or is a multi-component coating based on Parylene C, SiO₂, PDMS and ceramic. Parylene-type materials and coatings containing the same are known to the skilled person in the art. It is noted that the addition of a coating (28) may reduce—or even avoid—sharp edges of FPCB. Furthermore, a coating (28) may give the FPCB (2) a smooth structure and/or surface and protects the components (5b-h), such that they are not directly exposed to the living body.

When the electrodes (3, 4) are integrated into the FPCB (2), the first conductive material layer (21) of the FPCB (2) forms the electrodes (3, 4) Hence, most of the surface area of the monitor (1) is removed, e.g. etched away, while the remaining area of the layer (21) acts as electrodes (3, 4). The layer (21) is connected by a vertical interconnected access (via) with the signal layer (23), which itself is connected to the main circuit (5). Hence, the layer (21) measures biosignals within the living body. They are then transported through the via to the signal layer (23), optionally amplified and/or filtered by the preamplifier or buffer (3a), and further transported to the main circuit (5) for processing.

In one embodiment, the first conductive material layer (21) is integrated into, i.e. it is, the signal layer (23), wherein a specific area of the layer (23), e.g. of the size of a typical electrode (3, 4), is not covered by the dielectric layer (22) to provide proper electrical contact to tissue which surrounds the monitor (1).

In another embodiment, the first conductive material layer (21) is integrated into, i.e. it is, the signal layer (23), wherein a specific area of the layer (23), e.g. of the size of a typical layer (21), i.e. electrode, is covered by the dielectric layer (22) and the signal layer (23) is therefore capacitively coupled to the skin.

The first conductive material layer (21) may be laser-machined and/or coated to provide a 3-D pattern for a better electrical contact to skin and thus to obtain even a better signal quality, e.g. an improved signal-to-noise ratio. Non-limiting, suitable materials to coat the layer (21) are electrically conductive materials and include silver (Ag), gold (Au), copper (Cu), electroless nickel immersion gold (ENIG), iridium-platinum

Docket No. 1160.009 (Ir—Pt), iridium dioxide (IrO₂), titanium nitride (TiN), and/or polymers such as poly-3,4-ethylendioxythiophen (PEDOT) and silver-polydimethylsiloxane (Ag-PDMS). The thickness of such a coating may be between 0.05 μm and 1 μm, measured with X-ray according to DIN ISO 3497 or—if unsuitable for the specific case—scanning electron microscopy (SEM). The skilled person can make the proper selection.

The conductive material layer (21) and the one or more signal layers (23, 26) may be of the same or of a different material. Suitable conductive materials for the layers (21, 23, 26) are known to the skilled person. Non-limiting, but preferred materials for the conductive layers (21, 23, 26) include Cu, Au, Ni, Cr, Pd, Al, Ag, Sn, Pt, Ir—Pt, and/or electrically conductive polymers such as PEDOT or Ag-PDMS. The thickness of the layer (21) is preferably between 5 μm and 50 μm, in particular between 10 μm and 30 μm. The thickness of the layer (23) is preferably between 5 μm and 50 μm, in particular between 5 μm and 20 μm. And the thickness of the optional layer (26) is preferably between 5 μm and 50 μm, in particular between 10 μm and 40 μm, measured with X-ray according to DIN ISO 3497 or—if unsuitable for the specific case—scanning electron microscopy (SEM). The skilled person can make the proper selection.

The first and the further dielectric layers (22, 25) separate the conductive layers (21, 23, 26) from each other. Suitable dielectric materials for the layers (21, 23, 26) are known to the skilled person. Non-limiting, but preferred materials for the dielectric layers (22, 25) include liquid-crystal polymer (LCP) and/or polyimide (PI). LCP—as example—provides a number of advantageous properties, including biocompatibility, high mechanical flexibility and strength, good dielectric characteristics, multilayer circuit capabilities, high compatibility to the solder mask material, high durability, very low water absorption, excellent high-frequency electrical properties and thus it is suitable for RF applications and is chemically inert. Furthermore, LCP allows to cut out arbitrary forms from a sheet of FPCB, e.g. by laser cutting.

The thickness of the layer (22) is preferably between 10 μm and 200 μm, in particular between 25 μm and 100 μm, and the thickness of the layer (25) is preferably between 5 μm and 50 μm, in particular between 5 μm and 20 μm. And the thickness of the optional layer (26) is preferably between 10 μm and 100 μm, in particular between 15 μm and 50 μm, measured with X-ray according to DIN ISO 3497 or—if unsuitable for the specific case—scanning electron microscopy (SEM). The skilled person can make the proper selection.

The optional adhesive layer (24) adheres typically a conductive layer (21, 23, 26) to a dielectric layer (22, 25). Depending on the specifically used conductive layers (21, 23, 26) and dielectric layers (22, 25), and/or the process to manufacture the FPCB, the adhesive layer (24) might be omitted. Suitable adhesives for the layer (24) are commercially available and known to the skilled person in the art. A non-limiting, but preferred adhesive includes ULTRALAM™, in particular ULTRALAM™ 3908. He also can make the best selection. A typical thickness of the layer (24) ranges preferably between 5 μm and 50 μm, in particular between 10 μm and 40 μm, measured with X-ray according to DIN ISO 3497 or—if unsuitable for the specific case—scanning electron microscopy (SEM). The skilled person can make the proper selection.

The optional solder mask layer (27) forms—when present—the final layer of the FPCB (2) and thus covers and protects the layer underneath. In case the latter is e.g. a dielectric layer (22, 25) with sufficient resistance to the environment, the layer (27) may be omitted. Suitable materials for the layer (27) are commercially available and known to the skilled person in the art. He also can make the best selection. A typical thickness of the layer (27) ranges preferably between 5 μm and 50 μm, in particular between 10 μm and 40 μm, measured with X-ray according to DIN ISO 3497 or—if unsuitable for the specific case—scanning electron microscopy (SEM). The skilled person can make the proper selection.

The thickness of the FPCB (2) in total—measured in the vertical to the diameter of the open-circular shape according to DIN 50986—may vary from 0.05 mm to 4 mm, preferably from 0.15 to 2 mm.

The monitor (1) comprises a main circuit (5) to measure, to store and to transmit, i.e. read out, the recorded data. Since the main circuit (5) is integrated into the monitor (1), and thus into the FBCB, no cables and thus no connections are required. Hence, the monitor is robust and not prone to failure or fractures.

In a preferred embodiment, the main circuit (5) of the monitor (1) comprises a base (5a), an amplifier (5b), a controller (5c), optionally a battery (5d), a memory (5e), a transmitter or transceiver (5f), and optionally a DC-restorer (5g), and/or a feedback circuit (5h). Thereby the transmitter or transceiver (5f) can be powered by the battery (5d) of the main circuit (5), or the transmitter or transceiver (5f) can be powered by an external power source, thus making the battery (5d) of the circuit (5) redundant. Alternatively, or in addition, the transmitter or transceiver (5f) may be used to recharge the battery (5d) of the main circuit (5). In this case, the transmitter or transceiver (5f) most typically comprises a coil.

Preferably, the main circuit (5) is affixed to the sensing electrodes (3), i.e. to the layer opposite the first conductive material layer (4), which will contact the surrounding tissue.

Alternatively—or in addition —one or more sensing electrodes (3) comprise a preamplifier or a buffer (3a), thus, to become a so-called active electrode. Hence, the recorded signals from the first conductive material layer (21) are preamplified nearby, i.e. as close as possible, to the measurement point. This leads to lower susceptibility to magnetic or electrical field coupling and thus to a finally higher signal quality.

The amplifier (5b), the controller (5c), the optional battery (5d), the memory (5e), the transmitter or transceiver (5f) and the optional DC-restorer (5g), and/or the optional feedback circuit (5h) of the main circuit (5) are connected to at least one of the signal layers (23, 26) of the base (5a). Although the signal layer (26) may be covered with the optional solder mask layer (27), it may well be advantageous to further coat the components (5b-h) with a dielectric coating (28), such as coating including a Parylene-type material. It is noted that the layers (26, 27) and the coating (28) are arranged on side of the FPCB (2) which is opposite to the electrodes (3, 4).

The components (5b-h) are commercially available components and known to the skilled person in the art. He is well capable of making the best selection. Furthermore, he knows how to properly assemble and connect them to at least one of the signal layers (23, 26) to result in a main circuit (5) with optimized performance.

The amplifier (5b) amplifies the measured, analog signal for proper signal processing. A non-limiting example of a suitable amplifier (5b) is a battery-powered single-supply instrumentation amplifier with a gain of about 20 dB, a 3 dB bandwidth of 0.5 Hz-250 Hz and a CMRR of about 100 dB.

The controller (5c) converts the—optionally amplified—analog biosignals to digital signals, provides optional filtering and stores that signal in the memory (5e) and/or transmits it via the transmitter or transceiver (5f) to an external receiver. A non-limiting example of a suitable controller (5c) includes a 16-bit CPU, inputs and outputs, 128 KB non-volatile memory, 8 KB RAM and a 12-bit analog-to-digital converter with 10 channels, using a system clock of 12 MHz.

Preferably, the read-out data are deleted from the memory (5e) of the monitor (1). Alternatively, or in addition, the battery (5d) of the monitor (1) is recharged, preferably using RFID, such as NFC.

The battery (5d) provides the various components of the main circuit (5) sufficient electric energy. A non-limiting example of a suitable battery (5d) includes a lithium-ion battery with 4.2 V nominal voltage, having a capacity of 2000 mAh.

The memory (5e) stores the acquired and/or processed data. A non-limiting example of a suitable memory (5e) includes a non-volatile NAND flash, having a capacity of e.g. 8 GB.

The measured and stored data are transmitted by the transmitter or transceiver using wireless communication and read out/received by an external reader. Reading out the data is performed typically on demand, based on a predefined interval or by a physician, and/or permanently by a wireless connection to an external computer, smartphone, or other suitable receiver. The transmitter or transceiver (5f) may comprise a coil enabling wireless power transmission, i.e. wireless power transfer (WPT), wireless energy transmission (WET) or electromagnetic power transfer, most typically making use of an electromagnetic field. Thus, the battery (5d) of the main circuit (5) may be recharged via the transmitter or transceiver (5f). A non-limiting example of a suitable transmitter or transceiver (5f) includes a compatible interface to the controller (5c), a Bluetooth® Low Energy or wireless module using another low-power network technology, RFID, and/or optical wireless communication such as NIR, and an antenna.

The DC-restorer (5g) removes the offset from the amplified biosignals, introduced by electrode and/or conductor offset potentials, and therefore keeps the output signal in the required common mode range of the amplifier (5b). A non-limiting example of a suitable DC-restorer (5g) includes an inverting integrator opamp circuit.

The feedback circuit (5h) forces the body potential to a favorable circuit potential, e.g. the mid-supply voltage as well as actively suppresses the common mode voltage by negative feedback on the body, and hence at the input of the amplifier. A non-limiting example of a suitable feedback circuit (5h) includes an inverting low-pass filter opamp circuit.

### The Implantation Tool (6)

The implantation tool (6) of the kit according to the invention exhibits an open-circular shape to receive the open-circular monitor (1) reversibly. The implantation tool (6) includes a

base (61), which is suitable to accommodate the monitor (1) reversibly, handle (62), which is connected to the base (61), to hold and position the implantation tool, and

slider (63), which is insertable into the base (61) reversibly and thus capable to push the accommodated monitor (1) out of the implantation tool (6) to the final position,

wherein the base (61) includes a base bottom (61a), base sidewalls (61b) and a base surface which is at least partially open to allow the slider (63) to slide along the base bottom (61a) to push the accommodated monitor (1) out of the base (61). Hence, the base (61) of the implantation tool (6) has an open end to allow the insertion as well as the removal of the monitor (1). Furthermore, the base bottom (61a), the base sidewalls (61b) and the base surface are preferably arranged along the open-circular shape of the implantation tool (6).

The implantation tool (6) according to the invention is dedicated to implant the monitor (1) according to the invention easily under the skin of a living body. Hence, its base (61) may contain stabilizer layers on the side to receive and hold i) the slider (63) as well as ii) the monitor (1). The base (61) has preferably an open and a closed end, wherein the handle (62) may be positioned to act also to close the one end. Most typically, the slider (63) and the monitor (1) can be inserted into the base (61) from the open end, wherein the stabilizer layers on the side avoid a mal-placement of the slider (63) and the monitor (1). When the slider (63) is inserted first, followed by the monitor (1), the latter can be pushed out, e.g. implanted, by moving the slider (63). The handle (62) is connected to the base (61), to hold and position the implantation tool.

When in use, the handle (63) is connected to the base (61), to hold and use the implantation tool (6) properly.

The base (61), the handle (62) and the slider (63) of the implantation tool (6) may be made of the same or of different materials, wherein the material or materials are preferably selected from metal such as stainless steel, titanium, nickel-titanium, Cobalt-Chrome, and/or alloys; and/or synthetic materials such as polylactide (PLA), polyglycolide (PGA), poly(trimethylene carbonate) (PTMC), poly(p-dioxanone) (PDO), polyurethane (PUR); fluoropolymers such as polytetrafluoroethylene (PTFE) and polytetrafluoroethylene (PTFE); ultrahigh molecular weight polyethylene (UHMWPE), and/or polyethylene terephthalate (PET) wherein the implantation tool (6) may be coated with the dielectric coating (28).

The implantation tool (6) may be made with methods known to the skilled person in the art. Non-limiting examples include bending, casting, hammering, embossing, forging, punching, drawing, rolling, and/or 3-D printing.

### Process to Make the Monitor (1) and the Monitor (1) Obtainable According to Said Process

The process to make the monitor (1) of the kit according to the invention is characterized in that

the at least two sensing electrodes (3), the base (5a) of the main circuit (5) and the optional ground electrode (4) are made from the same flexible printed circuit board (FPCB) (2),

the amplifier (5b), the controller (5c), the optional battery (5d), the memory (5e), and the optional transmitter or transceiver (5f), the optional DC-restorer (5g), and/or the optional feedback circuit (5h) are connected to the base (5a) and thus to the main circuit (5), preferably by soldering, welding, bonding, and/or gluing, and

optionally at least the side of the FPCB (2), which is opposite to the electrodes (3, 4), and onto which the components (5b-h) are most typically being mounted to, is coated with the dielectric coating (28). In many cases, however, it is preferred when all sides of the FPCB (2) are coated with the dielectric coating (28).

Thus, the FPCB (2) with the required architecture and includes the desired layers, e.g. the layers (21) to (27), is made by crimping said layers, etching away the dispensable portions to generate the conductor paths, bored or lasered to generate the vertical vias, plate through to connect conductive layers by vertical vias followed by cutting out the desired form of the FPCB (5), e.g. by laser cutting. The processes of making such suitable FPCB's and cutting out the desired form are known to the skilled person in the art. Furthermore—and if required—the layer (21) may be edged—except at the locations of the electrodes (3, 4).

Hence, the components (5b-h) are preferably arranged at the side of the FPCB (2) which is opposite to the electrodes (3, 4). And, upon coating said side with the dielectric coating (28), also the components (5b-h) are coated therewith. Applying the dielectric coating (28) onto the components (5b-h), and on e.g. the layer (26) or (27), may be carried out before and/or after cutting the FPCB (2).

These steps to make the monitor (1) may be made at any order.

In one preferred embodiment of the process, the monitor (1) comprises three or more, preferably 6 or more, in particular 12 or more, sensing electrodes (3), wherein the electrodes (3) are arranged on one and the same surface of the monitor (1) to form the corners of an equilateral, isosceles or right-angled triangle having an angle of between 60° and 90°, in particular between 75° and 90° in order to make use of Eindhoven's triangle, wherein the optional ground electrode (4) is arranged between two sensing electrodes (3), preferably on the same surface of the monitor (1).

Thus, the present implantable, flexible multi-lead cardiac monitor (1) obtainable according to the invention is received, i.e. monitor (1) obtainable according to the process to make the monitor (1) of the kit, i.e. the monitor (1) obtainable according to the process to make the monitor (1).

### Process to Monitor Biosignals

The process to monitor biosignals with the monitor (1) of the kit according to the invention, and/or the monitor (1) obtainable according to the process to make the monitor according to the invention, is characterized in that

the biosignals are measured, preferably continuously, with the sensing electrodes (3),

the thus obtained data are stored on the main circuit (5), in particular in the memory (5e) of the main circuit (5), wherein preferably all measured data are stored until the data are read out, and

the measured and stored data are read out via the transmitter or transceiver (5f), wherein the transmitter or transceiver (5f) comprises an antenna, using a reader by wireless communication, preferably by RFID, such as NFC, and/or optical wireless communication such as NIR.

In case the monitor comprises more than 3 electrodes (3), the monitor may be equipped with a suitable algorithm to select for ECG measurements the electrodes (3) which fit best Einthoven's triangle. Alternatively, the electrodes (3) are configured externally after implantation, or the signals from all electrodes (3) are measured and stored.

In a preferred embodiment, the process to monitor biosignals according to the invention further comprises that

the read-out data are deleted from the memory (5e) of the monitor (1), and/or

the battery (5d) of the monitor (1) is recharged, preferably using RFID, such as NFC,

wherein the transmitter or transceiver (5f) comprises an antenna. Recharging the battery (5d) of the main circuit (5) occurs preferably by wireless power transmission via the transmitter or transceiver (5f) wherein the transmitter or transceiver (5f) most typically comprise a coil.

### The Uses

The kit according to the invention is particularly suited—and thus preferably used—to implant the monitor (1) of the kit and obtainable according to the process of the invention to make said monitor (1) with the implantation tool (6) of the kit according to the invention under the skin, in particular under the skin of a living body.

The monitor (1) of the kit according to the invention and the monitor (1) obtainable according to the process of the invention to make said monitor (1) is particularly suited for—and thus preferably used for—long-term cardiac monitoring over years, in particular when the monitor (1) is implanted under the skin, preferably under the skin of a living body.

The living body is most typically a human or an animal, wherein the animal is preferably a mammal such as a monkey, dog, cat, horse, cow, donkey. Particularly preferred are humans, i.e. the human body.

Alternatively, or in addition, the recording of the biosignals is preferably used for long-term measurement of biosignals such as electrocardiography (ECG), in particular long-term ECG, electroencephalography (EEG), electromyography (EMG), and/or biosignals from plethysmography or impedance.

In a preferred embodiment, the monitor (1) is used to store the measured data at least until the data are read out. With other words: the monitor (1) is thus preferably used as a continuous recorder and not just as an event-triggered recorder. As such the monitor (1) is able to also store data e.g. minutes or hours before, minutes or hours after an arrhythmic event, as well as the arrhythmic event itself.

The implantation tool (6) of the kit according to the invention is particularly suited to—and thus preferably used to—accommodate reversibly the monitor (1) of the kit according to the invention and the monitor (1) obtainable according to the process of the invention to make said monitor (1).

Furthermore, the implantation tool (6) is particularly suited to—and thus preferably used to—implant the monitor (1) under the skin, in particular under the skin of a living body.

### LIST OF CITED REFERENCE SIGNS

1 implantable, flexible multi-lead cardiac monitor (1)
2 flexible printed circuit board (FPCB) (2)
   21 first conductive material layer (21)
   22 first dielectric layer (22)
   23 signal layer (23)
   24 adhesive layer (24)
   25 further dielectric layer (25)
   26 further signal layer (26)
   27 solder mask layer (27)
   28 dielectric coating (28)
3 sensing electrodes (3)
   3a preamplifier or buffer (3a)
4 ground electrode (4)
5 main circuit (5)
   5a base of main circuit (5a)
   5b amplifier (5b)
   5c controller (5c)
   5d battery (5d)
   5e memory (5e)
   5f transmitter or transceiver (5f)
   5g DC-restorer (5g)
   5h feedback circuit (5h)
6 implantation tool (6)
61 base (61)
   61a base bottom
   61b base sidewalls
62 handle (62)
63 slider (63)

The following figures present non-limiting embodiments, which are not restricting or narrowing the invention. These explanations are part of the description:

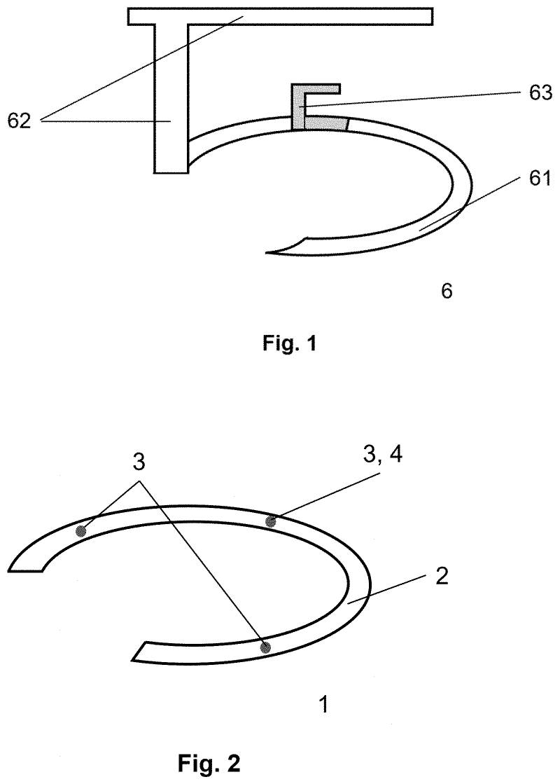

FIG. 1 illustrates a non-limiting and schematic scheme of an implantation tool (6) including the base (61) to accommodate the monitor (1), the handle (62) and the slider (63). The monitor (1) is designated to be placed between the slider (63) and the open end of the base (61).

FIG. 2 illustrates a non-limiting and schematic view of the monitor (1) includes the FPCB (2) with two sensing electrodes (3) and a third electrode (3, 4), which is either the optional ground electrode (4) or a third sensing electrode (3), arranged at the corners of an equilateral triangle.

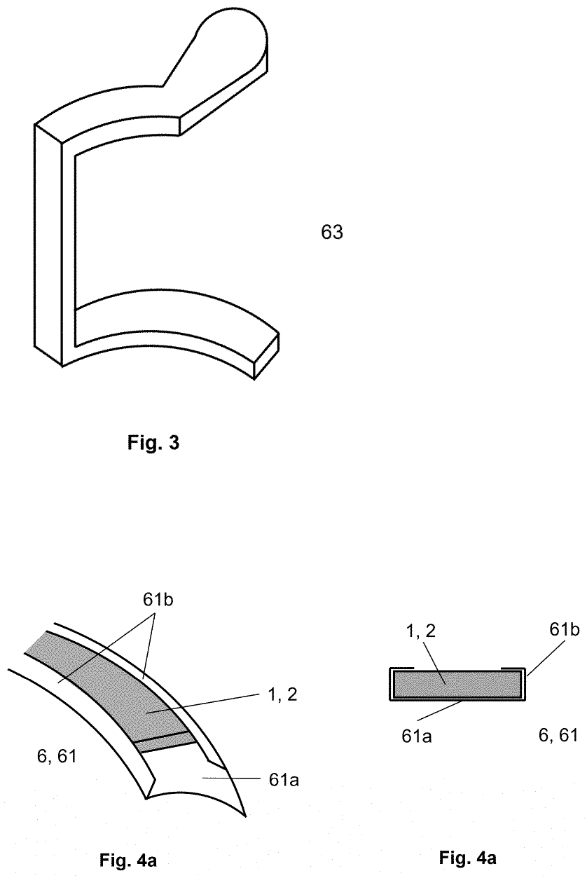

FIG. 3 illustrates a non-limiting embodiment of the slider (63) of the implantation tool (6). The lower portion is adjusted to be inserted into the base (61), next to the monitor (1).

FIG. 4a illustrates a non-limiting example of the open end of the base (61) of the implantation tool (6). The base (61) includes the base bottom (61a) and the base sidewalls (61b). The presented end-segment of the base (61) is designed to form a sharp, curved edge. The monitor (1) includes the FPCB (2) is inserted into the base (61) between the base sidewalls (61b), which are inwardly bent. The shown end of the monitor (1) may comprise a barbed hook (not shown), preferably of bioresorbable material to facilitate the removal of the implantation tool (6), including the base (61) and to anchor the monitor (1).

FIG. 4b illustrates the front-view at the open end of the base (61) from the implantation tool (6) includes another shape, wherein the base bottom (61a) comprises lateral base sidewalls (61b) with a small side cover. The lateral base sidewalls (61b) are in vertical position relative the horizontal base bottom (61a), thus forming a rectangular, curved base (61) of the implantation tool (6) which can easily receive reversibly the monitor (1) includes the FPCB (2). Most of the base surface of the base (61) is free of a cover to allow the slider (63) to be moved back and forth.

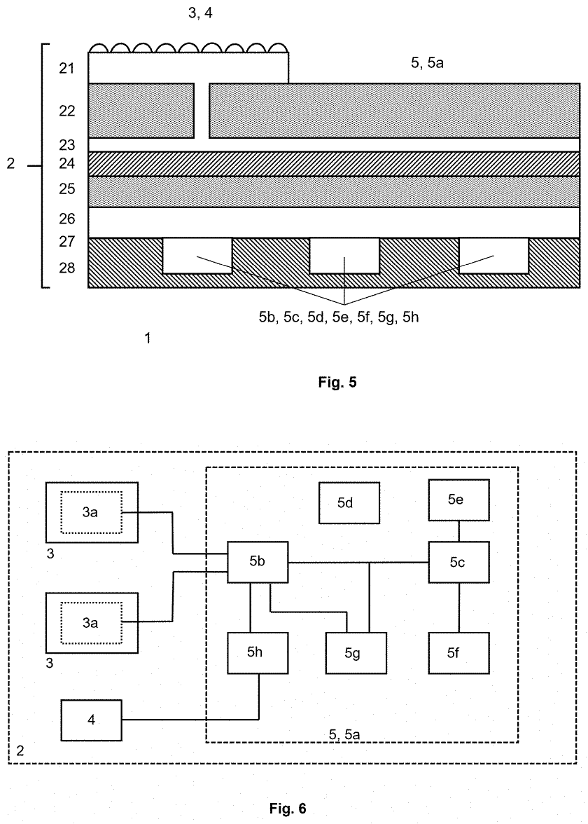

FIG. 5 shows an exemplary cross section of the FPCB (2) includes the signal recorder (2) includes an electrode (3, 4) and the main circuit (5) with the base (5a). All said components (3, 4, 5, 5a) are made from the one and same material, i.e. FPCB (2). The base (5a) of the main circuit (5) compose in this example of the first dielectric layer (22), the further conductive material signal layer (23), the adhesive layer (24), the further dielectric layer (25), the further optional signal layer (26) as well as of the solder mask layer (27).

On the left-hand side of the FPCB (2) are the various layers of an exemplary electrode (3, 4) visualized. The electrode (3, 4) comprises—in addition to the same FPCB-layers (22-27) from the base (5a)—the first conductive material layer (21), which is coated with a 3-D pattern for better contact to skin. The layer (21) is connected by a vertical interconnected access (via) to the further conductive material signal layer (23). Hence, the recorded biosignal is conducted from the layer (21) to the layer (23) of the electrodes (3, 4) along the layer (23) to the base (5a) of the main circuit (5), where it is further processed.

The amplifier (5b), controller (5c), the optional battery (5d), the memory (5e), a transmitter or transceiver (5f)—which may comprise a coil, and the optional DC-restorer (5g), and/or feedback circuit (5h), are—indicated as a number of rectangles—exemplary arranged at the optional further signal layer (26). Furthermore, these components (5b-h) as well as the layer (26) in-between—are exemplarily coated with the dielectric coating (28). It is noted that the optional dielectric coating (28) may also cover all other surfaces of the FPCB (2) except the electrodes (3,4), i.e. the layer (21).

FIG. 6 discloses a non-limiting block diagram of the main circuit (5) on the FPCB (2). The latter comprises the optional ground electrode (4), two sensing electrodes (3), each having a preamplifier (3a) integrated and the main circuit (5) with the base (5a), amplifier (5b), controller (5c), battery (5d), memory (5e), transmitter or transceiver (5f), DC-restorer (5g) and feedback circuit (5h). The ground electrode (4) and the sensing electrodes (3) are connected to the main circuit (5). The battery (5d) is connected (not shown) to each component (3, 4, 5, 5a-h) to provide them with the required electricity.

1. A kit for implanting an implantable, flexible multi-lead cardiac monitor (1) for recording biosignals when the monitor (1) is placed under the skin of a living body, the kit is comprising;
the cardiac monitor (1),
an implantation tool (6) for implanting the cardiac monitor (1) under the skin, and
optionally a surgical knife,
wherein the cardiac monitor (1) includes an open-circular shape, is based on a flexible printed circuit board (FPCB) (2) with at least two sensing electrodes (3) and optionally a ground electrode (4),
wherein the cardiac monitor (1) includes a main circuit (5) based on the FPCB (2), and wherein the monitor (1) is free of a casing; and
wherein the implantation tool (6) exhibits an open-circular shape to reversibly receive the open-circular cardiac monitor (1), the implantation tool (6) includes;
a base (61) to accommodate reversibly the cardiac monitor (1),
a handle (62) connected to the base (61), to hold and position the implantation tool, and
a slider (63), which is reversibly insertable into the base (61) and capable to push the cardiac monitor (1) out of the implantation tool (6) to a final position,
wherein the base (61) includes a base bottom (61a), base sidewalls (61b), and a base surface which is at least partially open to allow the slider (63) to slide along the base bottom (61a) to push the cardiac monitor (1) out of the base (61),
wherein the base bottom (61a), the base sidewalls (61b) and the base surface are arranged along the open-circular shape of the implantation tool (6).

2. The kit according to claim 1, wherein the main circuit (5) of the cardiac monitor (1) comprises a base (5a), an amplifier (5b), a controller (5c), optionally a battery (5d), a memory (5e), a transmitter or transceiver (5f), and optionally a DC-restorer (5g), and/or a feedback circuit (5h).

3. The kit according to claim 1, wherein the open-circular shape of the monitor (1) and of the implantation tool (6) includes a circumference of an angle of between 90° C. and 400° C., to allow placement of the at least two sensing electrodes (3) at comers of an equilateral, isosceles or right-angled triangle have an angle of between 60° C. and 90° C.

4. The kit according to claim 1, wherein:
the flexible printed circuit board FPCB (2) of the cardiac monitor (1) is a layered composite material comprising a first conductive material layer (21) capable to act as electrodes (3, 4), a first dielectric layer (22), a signal layer (23), optionally an adhesive layer (24), a further dielectric layer (25), an optional further signal layer (26), and/or an optional solder mask layer (27), wherein at least the side of the FPCB (2), which is opposite to the electrodes (3,4), may be coated with a dielectric coating (28); and/or
the base (61), the handle (62) and the slider (63) of the implantation tool (6) are made of the same or of different materials, wherein the material or materials are preferably selected from metal such as stainless steel, titanium, nickel-titanium, Cobalt-Chrome, and/or alloys; and/or synthetic materials such as polylactide (PLA), polyglycolide (PGA), poly(trimethylene carbonate) (PTMC), poly(p-dioxanone) (PDO), polyurethane (PUR); fluoropolymers such as polytetrafluoroethylene (PTFE) and polytetrafluoroethylene (PTFE); ultrahigh molecular weight polyethylene (UHMWPE), and/or polyethylene terephthalate (PET), wherein the implantation tool (6) may be coated with the dielectric coating (28).

5. The kit according to claim 1, wherein:
the outer diameter of the monitor (1) with the open-circular shape ranges from 3 to 20 cm; and/or
the inner diameter of the monitor (1) ranges from 2 to 18 cm.

6. The kit according to claim 1, wherein the electrodes (3) and the optional ground electrode (4) are integrated into the flexible printed circuit board (FPCB) (2), wherein the sensing electrodes (3) are arranged to form the corners of a polygon, in particular an equilateral, right-angled or equiangular polygon, and wherein the sensing electrodes (3) may comprise a pre-amplifier or buffer (3a).

7. The kit of claim 2, wherein:
the at least two sensing electrodes (3), the base (5a) of the main circuit (5) and the optional ground electrode (4) are made from the same flexible printed circuit board (FPCB) (2),
the amplifier (5b), the controller (5c), the optional battery (5d), the memory (5e), and the optional transmitter or transceiver (5f), the optional DC-restorer (5g), and/or the optional feedback circuit (5h) are connected to the base (5a) and thus to the main circuit (5), by soldering, welding, bonding, and/or gluing; and
optionally at least the side of the FPCB (2), which is opposite to the electrodes (3, 4) is coated with the dielectric coating (28).

8. The kit according to claim 7, wherein the monitor (1) comprises three or more sensing electrodes (3), wherein the at least three sensing electrodes (3) are arranged one and the same surface of the cardiac monitor (1) to form the corners of an equilateral, isosceles or right-angled triangle having an angle of between 60° C. and 90° C., in particular between 75° C. and 90° C. in order to make use of Eindhoven's triangle, and wherein the optional ground electrode (4) is arranged between two sensing electrodes (3), preferably on the same surface of the monitor (1).

9. An implantable, flexible multi-lead cardiac monitor (1) obtainable according to claim 7.

10. A process to monitor biosignals with the cardiac monitor (1) of the kit of claim 1, the process comprising the steps of:
continually measuring the biosignals with the sensing electrodes (3),
storing the data obtained from the sensing electrodes on the the memory (5e) of the main circuit (5), wherein all measured data are stored until the data are read out, and
reading out the measured and stored data via the transmitter or transceiver (5f), wherein the transmitter or transceiver (5f) comprises an antenna, using a reader by wireless communication, preferably by RFID, and/or optical wireless communication such as NIR.

11. The process of claim 10, wherein:
the read-out data are deleted from the memory (5e) of the monitor (1) and/or
that the battery (5d) of the monitor (1) is recharged using RFID.

12-15. (canceled)
